(19) United States
(12) Patent Application Publication
(10) Pub. No.: US 2009/0137043 A1
(43) Pub. Date: May 28, 2009

(54) METHODS FOR MODIFICATION OF POLYMERS, FIBERS AND TEXTILE MEDIA

(75) Inventors: Gregory N. Parsons, Raleigh, NC (US); Gary Kevin Hyde, Raleigh, NC (US); Joseph C. Spagnola, Raleigh, NC (US); Qing Peng, Raleigh, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: North Carolina State University

(21) Appl. No.: 12/277,696

(22) Filed: Nov. 25, 2008

Related U.S. Application Data
(60) Provisional application No. 61/004,370, filed on Nov. 27, 2007.

(51) Int. Cl. B32B 5/02; B05D 7/04; B32B 27/30; H01M 8/00; C12N 5/06; B01D 15/00; B32B 1/00; C23C 16/00; B01D 53/00; H01B 1/12; B01J 31/06; B32B 27/04

(52) U.S. Cl. 435/398; 427/212; 427/222; 428/221; 502/150; 442/118; 442/59; 252/501.1; 252/500; 210/502.1; 429/12; 264/81; 428/34.1

(57) ABSTRACT

The present subject matter relates to the modification of fibers by the growth of films by the Atomic Layer Epitaxy (ALE) process, which is also commonly referred to as Atomic Layer Deposition (ALD). The presently disclosed subject matter relates in particular to a process for the modification of the surface and bulk properties of fiber and textile media, including synthetic polymeric and natural fibers and yarns in woven, knit, and nonwoven form by low-temperature ALD.

### RELATED APPLICATIONS

The presently disclosed subject matter claims the benefit of U.S. Provisional Patent Application No. 61/004,370, filed Nov. 27, 2007, the disclosure of which is incorporated herein by reference in its entirety.

### GOVERNMENT INTEREST

The presently disclosed subject matter was made with United States Government support under Grant Nos. CHE-9876674 and CTS-0626256 awarded by NSF. Accordingly, the United States Government has certain rights in the presently disclosed subject matter.

### TECHNICAL FIELD

The presently disclosed subject matter relates to the modification of substrates, such as fibers, by the growth of films by the Atomic Layer Epitaxy (ALE) process, which is also commonly referred to as Atomic Layer Deposition (ALD). The presently disclosed subject matter relates in particular to a process for the modification of the surface and bulk properties of fiber and textile media, including synthetic polymeric and natural fibers and yarns in woven, knit, and nonwoven form by low-temperature ALD.

### BACKGROUND

New molecular-scale process technologies that can controllably and uniformly modify and reproduce arbitrary three-dimensional nano-architectures, including fiber mats and bundles, woven fabrics, and engineered polymer structures, will enable and facilitate new emerging fiber-based and textile products.

Due to the high curvature and heterogeneous nature of fibrous structures, existing surface modification technologies provide less than complete and uniform coverage of a textile material's surface. Current coating technologies for textiles often make use of liquid-based processes which require subsequent expensive drying or curing steps and conformality is typically less than ideal. During the chemical coating of textile goods, water is commonly used as the medium for applying the chemical treatments. The water must then be removed from the fiber or fabric during numerous rinsing and drying steps.

The type of fiber being used often determines the finishes and methods used to treat textile materials. In general, products comprising natural fibers require more processing when compared to synthetic fibers. Cotton fiber, the most used type of natural fiber, must undergo a series of preparation treatments to adequately clean the fibers for further processing. The different synthetic fibers can require very diverse finishing procedures. For example, polypropylene, a commonly used raw material in textile applications, is difficult to coat using wet treatment methods due to its hydrophobic nature.

Inorganic finishes, including coatings of silver, copper, and various metal oxides, have been used for many years in the textile industry. They are often applied using solution-based methods such as a pad-dry-cure process. Applications of textile materials treated with inorganic finishes range from increasing the conductivity of material such as carpet to reduce static electricity build-up to anti-bacterial finishes for medical face masks.

Coatings of inorganic materials, like those listed above, allow the creation of multifunctional textiles. Multifunctional textiles are materials that possess a combination of many different properties such as flame retardancy, water repellency, and antibacterial activity. These multifunctional textiles can be used for a number of different tasks, for example in such industries as medical, geotextiles and construction, upholstery, and filtration, to name a few. It is still desirable, however, for these coated textiles to still meet consumer demand in regards to comfort, ease of care, and health issues. Also, modified textile materials can protect against mechanical, thermal, chemical, and biological attacks, and at the same time offer improved durability and performance.

Different methods of deposition are used to create inorganic coatings on the surface of textile media. One technique involves the use of sol-gels, which are nanoparticulate materials, consisting of silica and metal oxides. Sol-gel coatings can be applied at room temperature using traditional textile application techniques such as pad application, dip coating, and spraying. Electroless plating can be used to deposit a catalytically active material, such as one containing palladium, onto a fiber surface from aqueous solution. The electroless plating method can require a pre-treatment step where the fiber or polymer surface is rendered hydrophilic in order to create uniform layers of the deposited metal.

Vapor phase processes, including atmospheric pressure plasma exposure, are currently used for textile modification and can be scaled to the rates required for high throughput processing. Plasma treatment, described, for example, in U.S. Pat. No. 4,550,578 can be used to functionalize the surface of textile materials, subsequently changing properties such as hydrophobicity or hydrophilicity. The uniformity of these methods is often not ideal, resulting in detrimental variations in material performance which can severely limit applications. For example, during the treatment of nonwoven fiber mats, it can be difficult for plasma treatment to uniformly coat the surface of the individual fibers within the mat.

Vapor phase methodologies that can substantially fully penetrate fibrous networks could be used, for example, to increase robustness, heat and fire resistance, and improve durability and cleaning, as well as enable electronic conduction, and catalytic and biocidal activity. Such methodologies represent a long-felt and ongoing need in the art.

### SUMMARY

In some embodiments, the presently disclosed subject matter provides a method for modifying the surface of a fiber-based substrate. The method can include introducing the fiber-based substrate into a reaction chamber, pulsing a vapor-phase precursor comprising an organic and/or inorganic component into the reaction chamber to create a partial atomic layer of the organic and/or inorganic component on the fiber-based substrate and create a first by-product species, purging the reaction chamber to remove excess of the vapor-phase precursor and the first by-product species, pulsing a vapor-phase reactant into the reaction chamber to complete the formation of an atomic layer of the desired material and create a second by-product species, purging the reaction chamber to remove excess of the vapor-phase reactant and the second by-product species, and repeating the pulsing and purging steps until the desired surface modification is achieved. In some embodiments the modification comprises a modification of surface energy.

In some embodiments, the presently disclosed subject matter provides a fiber-based substrate having a modified surface comprising a fiber-based substrate and a thin film formed on the fiber-based substrate. The thin film can be formed by the atomic layer deposition of a precursor comprising an organic and/or inorganic component and a vapor-phase reactant reactive with the organic and/or inorganic component. In addition, the thin film can modify the fiber-based substrate to have a desired surface. In some embodiments the fiber-based substrate has a modified surface energy.

In some embodiments, the presently disclosed subject matter provides a method for producing a high density amine-group functionalized surface on a fiber-based substrate. The method can include introducing the fiber-based substrate into a reaction chamber, pulsing a vapor-phase precursor comprising an inorganic component into the reaction chamber to create a partial atomic layer of the inorganic component on the fiber-based substrate and create a first by-product species, purging the reaction chamber to remove excess of the vapor-phase precursor, pulsing a vapor-phase ammonia into the reaction chamber to complete the formation of an atomic layer of the desired material and create a second by-product species, purging the reaction chamber to remove excess of the vapor-phase ammonia and the second by-product species, and repeating the pulsing and purging steps until the amine-group functionalized surface of the desired density is achieved.

In some embodiments, the presently disclosed subject matter provides a method for producing a uniformly hydrophilic surface on a fiber-based substrate. The method can include introducing the fiber-based substrate into a reaction chamber, pulsing a vapor-phase precursor comprising an inorganic component into the reaction chamber to create a partial atomic layer of the inorganic component on the fiber-based substrate and create a first by-product species, purging the reaction chamber to remove excess of the vapor-phase precursor and the first by-product species, pulsing a vapor-phase reactant into the reaction chamber to complete the formation of an atomic layer of the desired material and create a second by-product species, purging the reaction chamber to remove excess of the vapor-phase reactant and the second by-product species; and repeating the pulsing and purging steps until the uniformly hydrophilic surface is achieved.

In some embodiments, the presently disclosed subject matter provides a method for depositing polymer films on a fiber-based substrate. The method can include introducing the fiber-based substrate into a reaction chamber, pulsing a vapor-phase reactant comprising an organic monomer into the reaction chamber to create a partial atomic layer of the organic monomer on the fiber-based substrate and create a first by-product species, purging the reaction chamber to remove excess of the vapor-phase reactant and the first by-product species, pulsing a vapor-phase co-reactant comprising a complementary organic monomer into the reaction chamber to complete the formation of an atomic layer of the desired material and create a second by-product species, purging the reaction chamber to remove excess of the vapor-phase co-reactant and the second by-product species, and repeating the pulsing and purging steps until a desired polymer film is deposited.

In some embodiments, the presently disclosed subject matter provides a fabric (e.g., a polyolefin such as polypropylene) having a high density amine-group functionalized surface. The fabric can include a fiber-based substrate and an amine-group functionalized surface formed on the fiber-based substrate. The surface can be formed by the atomic layer deposition of a vapor-phase precursor comprising an inorganic component and a vapor-phase ammonia.

In some embodiments, the presently disclosed subject matter provides a modified fiber-based substrate comprising a fiber-based substrate and a polymer film formed on the fiber-based substrate. The polymer film can be formed by the atomic layer deposition of a vapor-phase reactant comprising an organic monomer and a vapor-phase co-reactant comprising a complementary organic monomer.

In some embodiments, the presently disclosed subject matter provides a method for depositing a hybrid organic-inorganic film on a fiber-based substrate. The method can include introducing a fiber-based substrate into a reaction chamber, pulsing a vapor-phase reactant comprising a first component comprising an organic component or an inorganic component into the reaction chamber to create a partial atomic layer on the fiber-based substrate and create a first by-product species, purging the reaction chamber to remove excess of the vapor-phase reactant and the first by-product species, pulsing a vapor-phase co-reactant comprising a second component comprising an organic or an inorganic component depending on the first component into the reaction chamber to complete the formation of an atomic layer of the desired material and create a second by-product species, purging the reaction chamber to remove excess of the vapor-phase co-reactant and the second by-product species; and repeating the pulsing and purging steps until the desired thickness of hybrid films is deposited.

In some embodiments, the presently disclosed subject matter provides a method for forming a free-standing micro- or nanostructure. The method can include introducing a fiber core into a reaction chamber, pulsing a vapor-phase precursor comprising an organic and/or inorganic component into the reaction chamber to create a partial atomic layer of the an organic and/or inorganic component on the fiber-based substrate and a first by-product species, purging the reaction chamber to remove excess of the vapor-phase precursor and the first by-product species, pulsing a vapor-phase reactant into the reaction chamber to complete the formation of an atomic layer of the desired material and create a second by-product species, purging the reaction chamber to remove excess of the vapor-phase reactant and the second by-product species; repeating the pulsing and purging steps until a desired thickness of a micro- or nanostructure is deposited, and removing the fiber core.

In some embodiments, the presently disclosed subject matter provides a method for prepare a micro- or nanostructure. The method can include providing a mold comprising a micro- or nanostructure, introducing the mold into an ALD reactor system, adjusting ALD process conditions to promote ALD reactant and product diffusion into and out of the mold, wherein a micro- or nanostructure is formed, and removing the mold.

It is an object of the presently disclosed subject matter to provide methods for modification of substrates, includes fibers and textile media.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIG. 1 is a schematic drawing illustrating the steps in the molecular layer deposition of a hybrid ZnO-Organic polymer layer;

FIG. 2 is a graph illustrating the mass gain of a hybrid ZnO-Organic polymer layer formed using the molecular layer deposition process of FIG. 1;

FIG. 3 is an x-ray photograph of a hybrid film formed using a molecular layer deposition process;

FIG. 4 is a graph illustrating the growth rate of aluminum oxide film deposited on cotton fabric using an ALD process;

FIG. 5 is a depiction of a detailed XPS scan of the Al2p peak for untreated cotton fabric and cotton fabric coated with Al₂O₃, illustrating the growth in intensity of the Al2p for increasing ALD cycles;

FIG. 6 is a graph illustrating the thickness of the walls of Al₂O₃ microtubes fabricated using ALD of PVA electrospun fiber templates;

FIG. 7 is a depiction of a XPS survey spectrum of cotton fabric coated with a thin film of TiN by the ALD process;

FIG. 8 is a graph demonstrating the effect of the number of ALD cycles on the static contact angle measurements of nonwoven polypropylene fabrics; and

FIGS. 9A-9C are schematic depictions of the use of ALD of Al₂O₃ in microfluidic channels inside a PDMS template. Starting with a PDMS template (FIG. 9A), the channels are coated by ALD (FIG. 9B). The PDMS can then be removed, resulting in an Al₂O₃ based microfluidic structure (FIG. 9C).

### DETAILED DESCRIPTION

The presently disclosed subject matter relates generally to methods for modification of substrates, such as fibers and textile media. The nanoscale conformality of Atomic Layer Deposition (ALD) processes makes them an attractive method for the coating of complex fibrous structures. The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

All publications, patent applications, patents, and other references mentioned herein are incorporated by reference in their entirety.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims.

Unless otherwise indicated, all numbers expressing quantities of reagents, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of mass, weight, concentration or percentage is meant to encompass variations of in one example ±20% or ±10%, in another example ±5%, in another example ±1%, and in still another example ±0.1 % from the specified amount, as such variations are appropriate to perform the disclosed methods.

The terms “fiber” and “fiber-based substrate” as used herein, are meant in their broadest sense to encompass all materials having a fibrous structure. For example, any polymer, fiber or textile material of a continuous shape is encompassed within the meaning of the terms fiber and fiber-based substrate as they are used herein. Accordingly, the fiber and fiber-based substrates of the presently disclosed subject matter include both synthetic and natural fibers as well as fiber-based materials produced by natural or synthetic approaches, such as but not limited to, cotton fibers and fabrics, protein-based fibers such as silk, elastomeric polymers and fabrics (e.g., polyolefins such as polypropylene) and polyvinyl alcohol polymers and fabrics. The fabrics of the presently disclosed subject matter include both woven and non-woven fabrics, and include, for example, a woven cotton fabric comprising yarns made up of many cotton fibers of different sizes and shapes.

As used herein the terms “micro-” and “nano-” have the meaning that would be ascribed to them by one of ordinary skill in the art. In some embodiments, these terms can refer to a structural feature having a dimension ranging from about 10 microns to about 1 nanometer (nm) in size. In some embodiments, the structural feature has a dimension ranging from about 10 microns to about 1 micron in size. In some embodiments, the structural feature has a dimension ranging from about 1 micron to about 100 nm in size. In some embodiments, the structural feature has a dimension ranging from about 100 nm to about 1 nm in size.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

### II. General Considerations

Atomic Layer Deposition (ALD), which can sometimes be referred to as Atomic Layer Epitaxy (ALE), Atomic Layer Chemical Vapor Deposition (ALCVD), or Molecular Layer Deposition (MLD), is a process, described, for example, in U.S. Pat. No. 4,058,430, for the fabrication of thin films. In ALD, film growth comprises a repeated binary sequence of self-limiting reactant adsorption and reaction steps. During the process, the self-limiting nature of the precursor adsorption results in material being built up as a series of atomic layers. The precursor molecules react with available surface groups, creating a saturated surface. After excess precursor is removed from the vapor phase by a purge gas (e.g., Ar), the reactant gas is subsequently pulsed onto the substrate, where it reacts with the adsorbed precursor layer to form a layer of the target film-forming material. Since no gas phase reaction occurs, the target film is grown layer-by-layer on the substrate. Therefore, the film thickness is directly controlled by the number of reactant exposure cycles used. The self-limitation of the ALD process allows increased conformality of ALD films on various substrates. Due to the fact that surface saturation occurs on all surfaces, conformality can be achieved for very high aspect ratio substrates. The partial reaction of the precursor in each deposition cycle differentiates ALD from more common chemical vapor deposition (CVD) processes and provides ALD an ability for high precision film formation.

Films can also be deposited by ALD at low temperatures. A process for low temperature ALD is described, for example, in U.S. Pat. No. 6,090,442. Low temperature ALD could be considered deposition at temperatures less than 200° C. One representative material for ALD is aluminum oxide (Al₂O₃). Aluminum oxide has many desirable traits such as strong adhesion to various surfaces, good dielectric properties, and chemical and thermal stability.

### III. Representative Embodiments

The presently disclosed subject matter relates generally to the production of thin films by an atomic layer deposition (ALD) process. Further, in some embodiments, the presently disclosed subject matter more particularly relates to the production of conformal, uniformly thin films with precise thickness and composition control over large scales. During a typical ALD process, a substrate can be placed within a reaction chamber where the substrate can be maintained at a suitable deposition temperature. For instance, the temperature can approach room temperature, depending on the reactant chemicals and reaction conditions employed.

Film growth during ALD comprises of a set of sequential, self-limiting deposition processes that operate on the principle of alternating, saturating surface reactions. These surface reactions can be implemented by directing gaseous or vaporized source materials alternately into the reactor and by purging the reactor with an inert gas between the precursor and reactant pulses. The vapor-phase precursor forms a (sub)monolayer of the precursor material on the substrate surface as the precursor molecules react with available surface groups, creating a saturated surface. Excess precursor can be removed by introducing an inert purge gas, such as Ar. The vapor-phase reactant can then be introduced into the reaction chamber where it can react with the adsorbed precursor layer to form a thin film of the target material. Excess of the reactant material and by-products of the surface reactions can be removed by the pulsing of the purge gas. The ALD process is based on controlled surface reactions of the precursor and reactant chemicals. The steps of pulsing and purging can be repeated in a sequential fashion, allowing the thickness of the deposited film to be accurately controlled by the number of cycles the process is repeated. The alternating, stepwise nature of the ALD method can prevent gas-phase reactions during the process.

The ALD technique can permit the controlled deposition of thin films of up to about 0.5 nm per cycle, providing a method for precise control over coating thickness. This growth rate can be adjusted by a changing a number of parameters in the ALD process. Sample films thicknesses and operating parameters such as temperature, pressure, and reactant times are described in the provided examples.

A wide variety of materials can be deposited by ALD including metals, metal oxides, metal nitrides, polymers, organic-inorganic hybrid layers, and other materials. Specifically, the deposition of certain materials, such as Al₂O₃, TiO₂, TiN, and SiO₂ for example, can be conducted by ALD at relatively low temperatures (e.g., less than about 150° C.), thereby limiting thermal damage to temperature-sensitive materials. In addition to creating thin films of one material, ALD can also be used to create microstructures and nanostructures, such as but not limited to nanolaminates of different materials.

The presently disclosed subject matter pertains in some embodiments to the use of ALD as a method of coating textile materials with thin films of materials, such as but not limited to metals and metal oxides. The ability of ALD to deposit materials at low temperatures makes it particularly suited for coating of thermally sensitive substrates such as fibers. The use of self-limiting reactant adsorption processes enables achievement of fully conformal functionalization of textile fibers of any continuous shape. This allows for well-controlled surface energy modification.

The presently disclosed subject matter aims to eliminate the problems with related art as noted above and to provide novel methods of producing thin films by ALD on the surface of substrates, such as a surface of textile materials and polymer fiber media.

In some embodiments, a method for depositing ultrathin conformal coating on textile materials comprising conducting a sequence of two or more self-limiting reactions at the surface of the textile materials to form ultrathin conformal coatings bonded to the surface of the materials is provided. A self-limiting reaction occurs between gas phase precursor molecules and a solid surface. Self-limiting reactions allow specific growth rates to be achieved for a given set of process conditions. The reaction stops once all of the surface sites on the substrate have reacted. One ALD cycle results in a repeatable amount of film growth without the production of extra film. The products and remaining reactant are then removed from the system. The textile materials that can be coated can comprise fibers, yarns, and fabrics either natural, man-made, or combinations of the two, and the textile materials can be in woven, knit, or nonwoven form.

The presently disclosed subject matter also provides in some embodiments a textile material coated with inorganic, organic, or hybrid organic/inorganic materials, such that the fibrous components of the textile material have an ultrathin conformal coating on their surface.

In general, the presently disclosed subject matter is suitable for depositing thin films of materials, such as but not limited to metals and metal compounds, at low temperatures. For the presently disclosed subject matter, an ALD process can comprise a set of sequential reactions carried out within a closed system at a pressure ranging from 0.5 Torr to 10 Torr. The thin films can be deposited at a range of temperatures from 25 to 200° C. The reaction temperature used can be determined by the nature of the substrate that is used and the characteristics of the thin film desired. When working at lower temperatures, precursors and reactants of sufficient reactivity, such as trimethylaluminum and water, can be used.

Examples of materials that can be deposited to form ultrathin conformal coatings include, but are not limited to, aluminum oxide, titanium nitride, and cobalt.

The resulting ALD-grown conformal thin films can be utilized, for example, as surface coatings for the creation of fabrics, such as cotton fabrics, with improved moisture barrier properties, as well as enabling layers for the functionalization of polypropylene and other fiber-based materials.

Inorganic materials are of particular interest as thin film coatings for fiber and textile materials. Coatings that are of particular interest are those which (1) improve stability of a material for mechanical, chemical, photo-chemical, or thermal destruction, (2) improve water, oil, and soil repellency properties of a material, (3) exhibit unique light absorption and emission properties in the UV and IR regions, (4) change the electrical conductivity of a material, (5) control release or immobilization of various active species. In addition, fibers and textile materials that are modified by such films can exhibit increased yield strength, reduced strain at yield stress, increased elastic modulus, increased fiber toughness, as well as increased wettability. It will be recognized that many materials are useful for more than one of these applications and that inorganic thin films will be useful for other applications not described here.

Examples of inorganic materials that can change the physical properties of fiber and textiles materials include, for example, various oxides, nitrides, and non-oxide materials. Titanium dioxide is a specific example of an oxide that can influence many different properties. Titanium dioxide is a wide band-gap semiconductor and is known to be a good oxidizing agent for photo-excited molecules and functional groups, making it useful as a photocatalyst or sensor material. Fibers coated with a thin film of titanium dioxide could provide high surface area catalytic mantles. Aluminum oxide is another good example of a coating material that can be deposited using ALD. Aluminum oxide has many favorable traits including strong adhesion to different substrate surfaces, good dielectric properties, and good chemical and thermal stability.

Inorganic materials useful in the controlled release or immobilization of active species include, for example, titanium nitride, silver, and copper.

In some embodiments, a thin film can be produced on a fiber or textile substrate by a process comprising introducing a substrate into a reaction chamber, pulsing a vapor-phase precursor containing the desired inorganic component (e.g. Al(CH₃)₃) into the reaction chamber to create an atomic layer of a precursor on the substrate, purging the reaction chamber to remove excess vapor-phase precursor, pulsing a vapor-phase reactant (e.g. H₂O) into the reaction chamber, purging the reaction chamber to remove excess of the vapor-phase reactant and the by-products of the reaction between the precursor and reactant, and repeating the pulse and purge steps until a coating of the desired thickness is formed.

In some embodiments of the presently disclosed subject matter, the final structure of the thin film can comprise a combination of layers, such as but not limited to metal containing layers stacked on together. For example, a film comprising alternating layers of Al₂O₃ and TiO₂ can be fabricated.

The substrate used can comprise any fiber or textile material of a continuous shape. In this regard, the shape of the fiber or textile material need not be limited to common cylindrical fibers or planar substrates. The ALD method can be used to create a conformal coating of individual fibers having complex shapes and surface topologies (e.g., corrugated substrate, non-woven web). For example, a woven cotton fabric comprising yarns made up of many cotton fibers of different sizes and shapes can be used as a substrate. In another example, the textile material can be formed from melt blown polyolefin (e.g. polypropylene) nonwoven fiber mats, wherein molten polymer is drawn through a quenching medium and hot air causes attenuations and fibrillation, creating fibers having large variability in diameter (e.g., 0.2 to 20 microns). In yet another example, the textile material can be spun bond polyolefin nonwoven fiber mats, where molten polymer is extruded through a spin pack, quenched by cold air, lengthened and tangled by warm air, and calendared and compacted by rollers. The fiber mats produced in this manner can have a more uniform size distribution, with fibers having diameters of approximately 12 to 50 microns.

The ALD method described above can be performed at low temperatures ranging from 25 to 200° C., depending on the nature of the fiber or textile material being used. Synthetic fibers may have a range of melting temperatures depending on the polymer they are constructed from. For example, polypropylene fibers have a melting point of 150° C. Natural fibers, such as cotton fiber, have a burning point rather than a melting point and start to degrade at temperatures over 100° C. Precursors and reactants of sufficient reactivity, such as trimethylamines, can be used in order to improve deposition at low temperatures. The ALD process can be carried out at low temperatures in order to prevent degradation of the fiber and textile substrates. Fiber and textile materials are often very sensitive to temperature changes, resulting in changes to their performance capabilities. Therefore, it can be advantageous to keep reaction temperatures as low as possible. The reaction temperature can be increased or decreased depending on the nature of the particular fiber or textile substrate being used. In some embodiments the ALD process of the presently disclosed subject matter can be performed in a chamber under a pressure ranging from 0.5 Torr to 10 Torr. The examples provided illustrate different reaction conditions that may be used to coat polymer and fiber based substrates.

The ALD process as described herein can provide modified dose and purge times based on the nature of the textile and fiber substrates that can be used. Depending on the characteristics of the substrate, longer dose and purge times can be necessary to completely saturate the surface. For example, a dense, layered, nonwoven fiber web would require longer dose and purge times when compared to a loosely knit fibrous structure. In addition, reactants generally require longer times to diffuse into the porous fiber samples (such as cotton fiber samples), possibly leading to a change in the growth rate. Thus, surface modification in accordance with the presently disclosed subject matter can include penetration into the bulk of the fiber.

Further, during each deposition cycle on a porous substrate, the precursor, reagent, and reaction products can travel through the winding fiber structure of the substrate to reach or be removed from the growth surface, and any precursor molecules remaining the fiber matrix after the gas purge step can lead to excess growth in the subsequent cycle. Also, the initial reactants can adsorb into the outer surface of the fibers, requiring more time before a uniform film is created. Examples of representative suitable dose and purge times are described in the Examples below.

The orientation of the fibrous or textile materials within the ALD reactor can be used to control the uniformity and conformality of the deposited thin films. For example, the arrangement of a substrate so that flow-through of the reactants is achieved can result in better uniformity and lower the amount of reactants required.

In some embodiments of the presently disclosed subject matter, the surface energy of fiber-based substrates can be modified. In some embodiments of the presently disclosed subject matter, a method is provided for modifying the surface energy of a fiber-based substrate comprising introducing the fiber-based substrate into a reaction chamber; pulsing a vapor-phase precursor comprising an inorganic component into the reaction chamber to create an atomic layer of the inorganic component on the fiber-based substrate; purging the reaction chamber to remove excess of the vapor-phase precursor; pulsing a vapor-phase reactant into the reaction chamber; purging the reaction chamber to remove excess of the vapor-phase reactant and the by-products of the reaction between the inorganic component and reactant, and repeating the pulsing and purging steps until the desired surface energy is achieved. For instance, the surface energy of the fiber-based structure can be modified to form a uniformly hydrophilic surface. A uniformly hydrophilic surface will demonstrate the same contact angle (<90°) over the surface of the sample.

In some embodiments, the fiber-based substrate includes but is not limited to cotton fiber, cotton fabric, woven cotton fabric, non-woven cotton fabric, protein-based fiber, polyvinyl alcohol fiber, polyvinyl alcohol fabric, woven polyvinyl alcohol fabric, non-woven polyvinyl alcohol fabric, polyolefin (e.g., polypropylene) polymer fiber, polyolefin fabric, woven polyolefin fabric, non-woven polyolefin fabric, polyethylene terephthalate fiber, polyethylene terephthalate fabric, woven polyethylene terephthalate fabric, non-woven polyethylene terephthalate fabric, polyamide fiber, polyamide fabric, woven polyamide fabric, non-woven polyamide fabric, acrylic fiber, acrylic fabric, woven acrylic fabric, non-woven acrylic fabric, polycarbonate fiber, polycarbonate fabric, woven polycarbonate fabric, non-woven polycarbonate fabric, fluorocarbon fiber, fluorocarbon fabric, woven fluorocarbon fabric, non-woven fluorocarbon fabric, glass fiber, glass fabric, woven glass fiber, and non-woven glass fabric. In some embodiments, the fiber-based substrate can be a planar surface, and in some embodiments a natural or synthetic polymer-based surface. In some embodiments, the fiber-based substrate can be a three-dimensional surface, in some embodiments a natural or synthetic polymer-based surface. In some embodiments, the polymer-based surface includes but is not limited to polyimide, polyethersulfone, cellophane, polydimethylsiloxane, and polytetrafluoroehtylene.

In some embodiments, an ALD process can be used to coat and modify low-cost polymer fibers to produce a surface that can be readily functionalized. For instance, in some embodiments, the modification of the surface energy of the fiber-based substrate can entail atomic layer deposition being used for surface treatment for fiber-based filters by depositing a material with a strong surface charge, and thereby providing an efficient and durable approach to enable surface functionalization. Referring to one specific example, a nonwoven fiber mat constructed of a synthetic polymer such as polypropylene, after coating with ALD, can become a low-cost and easy to handle filtration platform to enable a chosen chemical functionality, such as affinity ligands, to be bound to the surface with very high density. Such novel device platform materials can result in a wide variety of new applications, including blood purification, water decontamination, specialty nanoparticle, and nanotube collection, as well as chemical and bio-hazard detection systems. A particular example is the production of precision modified low-cost nonwoven fibers for use in targeted protein filtration and separation devices, such as a blood filtration device. Such devices can be effective at removing transmissible spongiform encephalopathies caused by prion proteins in contaminated blood supplies. Experiments have shown that the surface energy of the coated fibers can depend on the material used for coating, as well as the thickness of the ALD coating applied. The nonwoven fiber platform is an example of a complex surface topology, where the surface contour and appearance changes as one adjusts the scale of observation. Detailed examples of the use of ALD for modification of a synthetic fiber-based fibrous structure are provided in Examples 5-7.

Accordingly, in some embodiments, the presently disclosed subject matter provides a fabric having a high-density amine-group functionalized surface. In some embodiments the fabric comprises natural or synthetic materials. In some embodiments, the fabric can comprise a polyolefin such as polypropylene. In some embodiments the fabric can be a non-woven fabric. In some embodiments, the non-woven fabric filter can further comprise a bound affinity ligand. In some embodiments, the filter comprising a bound affinity ligand can be useful for removing transmissible spongiform encephalopathies caused by prion proteins from blood. In some embodiments, the filter comprising a bound affinity ligand can be useful for water decontamination.

Other possible applications include, but are not limited to, new formats and platforms for active electronic and energy conversion devices, as well as fuel cells, target-selective nano and biomolecule filtration and separation structures, tissue engineering scaffolds, and high performance engineered fibers and fabrics.

High surface area complex nanostructures are gaining interest in electronic systems. Examples include organic-based photovoltaic structures and novel fuel cell designs where increased surface area enhances the overall device efficiency. (See, for example, U.S. Pat. Nos. 3,969,163 and 7,160,424, the disclosures of which are incorporated herein by reference in their entirety) In some embodiments of the presently disclosed subject matter, highly uniform coating techniques such as ALD can allow modification of the surface functionality and composition within the complex nanostructure to broaden the applicability and reduce the fabrication cost of such device systems.

In some embodiments of the presently disclosed subject matter, manufacturing techniques are of interest that can modify fiber surface functionality, as well as the bulk properties within a woven fabric to protect against mechanical, chemical, biological and thermal exposure, and effectively repel undesirable foreign substances, while maintaining the benefits of light-weight breathable fabrics. (See, for example, U.S. Pat. Nos. 4,007,305, 4,623,574, 4,987,026, 5,298,303, and 6,187,391, the disclosures of which are incorporated herein by reference in their entirety) Inorganic insulator and metallic coatings on engineered fabrics are capable of meeting at least some of these objectives. Extending reactive systems and components to fabric platforms to produce catalytic mantles is another area of application.

In some embodiments of the presently disclosed subject matter, methods are provided for reproducibly converting the surface of fiber systems into inorganic material forms to, for example, significantly change the wetting properties of filters and other separation media, or enable template fabrication of hollow nanoscale needles, spheres, or other structures for bio-medical or tissue engineering applications. Also, in addition to surface chemistry, the wettability of a surface can be affected by the surface topography and roughness. For example a large contact angle observed for coated fibers can be ascribed to an increase in the fiber rigidity by the more incompliant inorganic coating, effectively reducing the total contract area between the fiber and the water droplet. For a super-hydrophobic material, a contact angle of greater than 120° is desired. Accordingly, the ability to conformally modify woven textile materials with near monolayer precision can provide new multifunctional textiles with properties and performance that deviate radically from current structured fabrics. As noted above, these multifunctional textiles can be used for a number of different tasks, for example in such industries as medical, geotextiles and construction, upholstery, and filtration, to name a few. In addition, these modified textile materials can still meet consumer demand in regards to comfort, ease of care, and health issues, and the modified textile materials can protect against mechanical, thermal, chemical, and biological attacks and offer improved durability and performance.

In some embodiments of the presently disclosed subject matter, methods are provided for surface modification of fiber webs using biocompatible materials such as TiN as a coating for implant materials including, for example, heart valves and orthopedics, due to the superior mechanical properties, corrosion resistance, and low cytotoxicity of TiN. TiN is often used as a hard, wear-resistant surface treatment, and it has been investigated as an antibacterial coating. The self-limiting film growth mechanism that is characteristic of ALD provides a technique to coat a wide range of substrates using conditions more favorable than other methodologies such as physical vapor deposition or plasma immersion ion implantation. Due to the nature of the process, self-limiting reactions allow for high precision of metallic and metal oxide deposition on the nano-scale. In addition, the use of ALD offers an environmentally friendly method for the formation of biocompatible materials. As a result, ALD processing can provide a valuable approach to control surface properties of fibers and other implant materials to promote preferred extracellular protein interactions for healthy cell adhesion and proliferation. See also Example 9 herein below.

In some embodiments of the presently disclosed subject matter, a method is provided for depositing polymer films on a fiber-based substrate comprising introducing the fiber-based substrate into a reaction chamber; pulsing a vapor-phase reactant comprising an organic monomer into the reaction chamber to create an atomic layer of the organic monomer on the fiber-based substrate; purging the reaction chamber to remove excess of the vapor-phase reactant; pulsing a vapor-phase co-reactant comprising a complementary organic monomer into the reaction chamber; purging the reaction chamber to remove excess of the vapor-phase co-reactant and the by-products of the reaction between the reactant and co-reactant, and repeating the pulsing and purging steps until the desired thickness of polymer films is deposited. In some embodiments, the polymer films are deposited on fiber-based substrates using a reactant and co-reactant comprising an organic monomer, and the reactant organic monomer and the co-reactant organic monomer have complementary end-groups to enable binary self-limiting reaction steps. In some embodiments, the complementary end-groups include but are not limited to end-groups such as aldehyde, anhydride, amine, ethyne and sulfide. In some embodiments, the reactant comprising the organic monomer can be pyromellitic dianhydride and the co-reactant comprising the organic monomer can be phenylene diamine. In some embodiments, the reactant comprising the organic monomer can be phenylene diamine and the co-reactant comprising the organic monomer can be phenylene dialdehyde. The examples provided above are a small representative subset of reactant and co-reactant molecules that can be used for deposition of polymer layers.

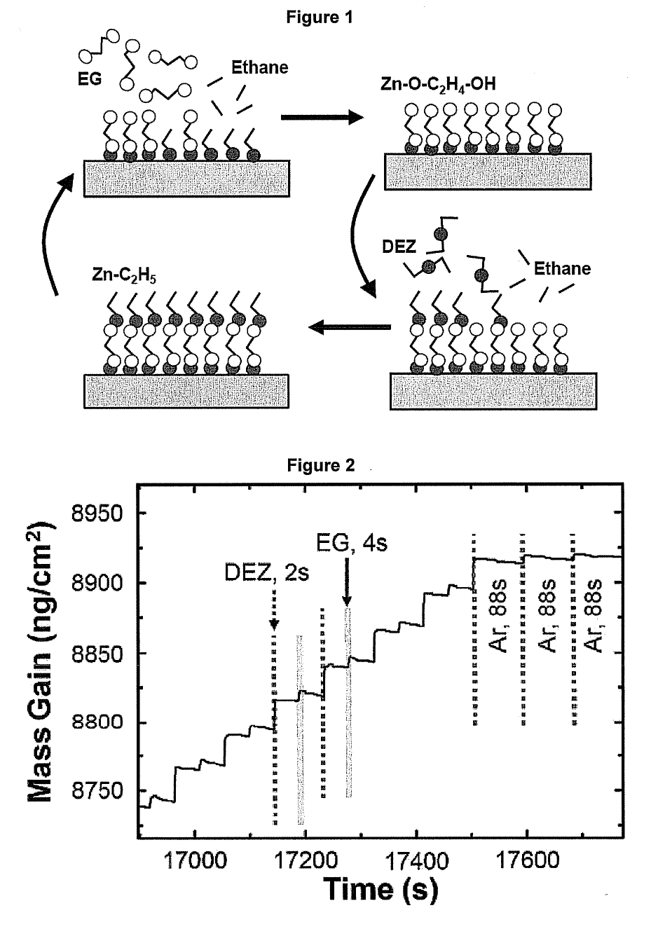

In some particular embodiments of the presently disclosed subject matter, methods are provided for atomic layer deposition of a hybrid organic-inorganic film on a substrate. The method can include introducing a fiber-based substrate into a reaction chamber, pulsing a vapor-phase reactant comprising a first component comprising an organic or an inorganic component into the reaction chamber, purging the reaction chamber to remove excess of the vapor-phase reactant, pulsing a vapor-phase co-reactant comprising a second component comprising an organic or an inorganic component (i.e., depending on the first component) into the reaction chamber, purging the reaction chamber to remove excess of the vapor-phase co-reactant and the by-products of the reaction between the reactant and co-reactant, and repeating the pulsing and purging steps until the desired thickness of hybrid films is deposited. If the reactant is an organic component, the co-reactant can be an inorganic component (or vice versa). For instance, FIG. 1 depicts an exemplary step-wise progression of these steps using ethylene glycol EG as the reactant and diethyl zinc DEZ the co-reactant. FIG. 2 shows the mass gain over time for the hybrid polymer film (e.g., (—OZnO—C₂H₄—)ₙ) formed in this manner.

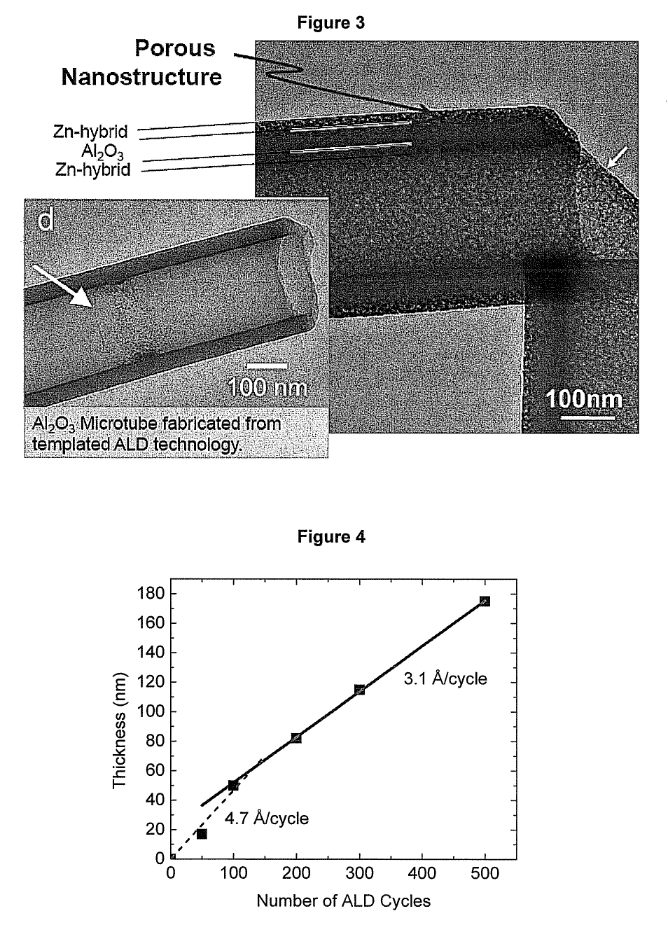

In some embodiments of the presently disclosed subject matter, methods are provided for atomic layer deposition for fabricating replica structures of both natural and synthetic fibrous systems. Replicate fiber structures can be created by performing ALD on various fiber formats (including cotton fiber formats), including single fibers, yarn bundles and woven fabrics, and subsequently removing the cotton fibers. With a sufficiently thick ALD coating, removal of the core fibers results in free-standing micro- and/or nanostructures (e.g., Al₂O₃ tubules) as fibers, “yarns” and woven structures. The resulting yarns and woven structures are surprisingly flexible and robust, even after the fiber core is removed. Only a very small number of cycles are needed to obtain measurable free-standing micro- and/or nanostructures. Such templated structures, developed from readily available woven or non-woven fiber and fabric materials can act as an inorganic base material for a range of advanced devices. Moreover, the ability to fabricate and manipulate free-standing materials that are less than 10 Å thick is a unique attribute of ALD that can be exploited for any of a variety of micro- and nanoscale applications. In other related embodiments, ALD processes can be used to form porous micro- and/or nanostructures. (See, e.g., FIG. 3) The porous nanostructure can have a controlled porosity based on desired properties for the structure.

In some embodiments of the presently disclosed subject matter applications are provided for micro- and nano-fluidics. Microfluidics is an enabling technology that makes possible the study of a range of biological and chemical systems. Typical applications include microliter to femtoliter chemical analysis and reaction, medical diagnostics, chemical and biochemical separation, and environmental monitoring. Moreover, microfluidics offers engineered structures with dimensions comparable to that of individual cells, organelles, and single biomolecules. Laboratory microfluidic systems are typically fabricated by casting polydimethylsiloxane (PDMS) against a mold and affixing it to a suitable flat surface. PDMS has several advantages over other materials, particularly its low cost and ease of fabrication. However, PDMS has significant limitations, especially in contact with organic media. A key problem with PDMS is that it is hydrophobic, so the channels are difficult to wet and they tend to bind hydrophobic bio-materials. The surface can be relatively inert, so that there is no available simple route for surface modification, although many methods to modify PDMS have been evaluated. Moreover, there is interest in moving from flat 2D system geometries to more complex 3D fluidic systems.

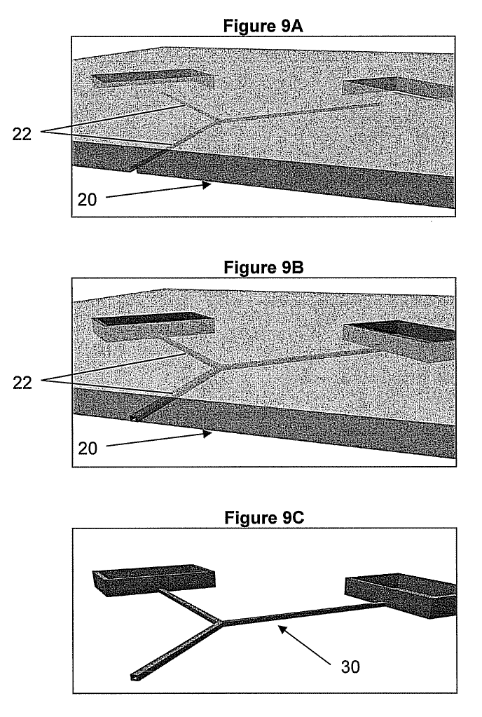

Accordingly, in some embodiments of the presently disclosed subject matter, methods are provided for preparing microfluidics structures. In some embodiments the methods involve material nucleation on a hydrophobic surface. In this manner, the ALD process can enable a structural characteristic (e.g., a dimension of a microfluidic channel) to be controlled at the atomic monolayer level. In some embodiments, an ALD process is performed in micro- or nanostructures (e.g., microfluidic channels) inside a mold, such as a PDMS template (see Example 8; FIG. 9A-C). Starting with a mold 20 (FIG. 9A), channels 22 can be coated using ALD (FIG. 9B). Mold 20 can then be removed, resulting in a micro- or nanostructure 30. In some embodiments, the micro- or nanostructure can be an Al₂O₃ based microfluidic structure (FIG. 9C).

Representative micro- and nanoscale systems and uses, including microfluidic systems and uses, are disclosed in the following published documents, which are incorporated herein by reference in their entirety: WO2005/101466, WO2005/084191, WO 2007/021755, WO/2007/021809, WO 2007/021810, WO 2007/021811, WO 2007/021812, WO 2007/021813, WO 2007/021815, WO 2007/021816, WO 2007/021817, and WO 2007/024485.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

Aluminum oxide (Al₂O₃) films were grown on cotton fabric substrates in a hot-wall viscous-flow tube reactor at temperatures ranging from 75° C. to 200° C. It was seen that at temperatures greater than 150° C., the cotton substrates became visibly discolored and physically brittle after the deposition process. It was found that 100° C. was an ideal operating temperature in that damage to the substrates was limited while still allowing adequate deposition of the Al₂O₃.

Trimethylaluminum (TMA) and deionized water were used as precursor and reactant, respectively, for the deposition of Al₂O₃. Reactant lines were heated to 60° C. in order to prevent condensation of the water reactant. Immediately before the deposition process, the substrates were placed in the reactor and heated in vacuum (5×10−7 Torr) to ˜100° C. and allowed to equilibrate for 60 minutes. To begin deposition, the reactor was flushed with argon and ambient temperature vapors of TMA and water were separately introduced into the reactor in pulses of 1 and 2 seconds respectively, with a 20 second Ar purge between each reactant exposure step. The TMA and water were carried into the reactor using Ar flow, and the Ar flow rate was constant at 100 standard cubic centimeters per minute (sccm). The operating pressure in the reaction chamber was fixed between 0.5 and 1 Torr.

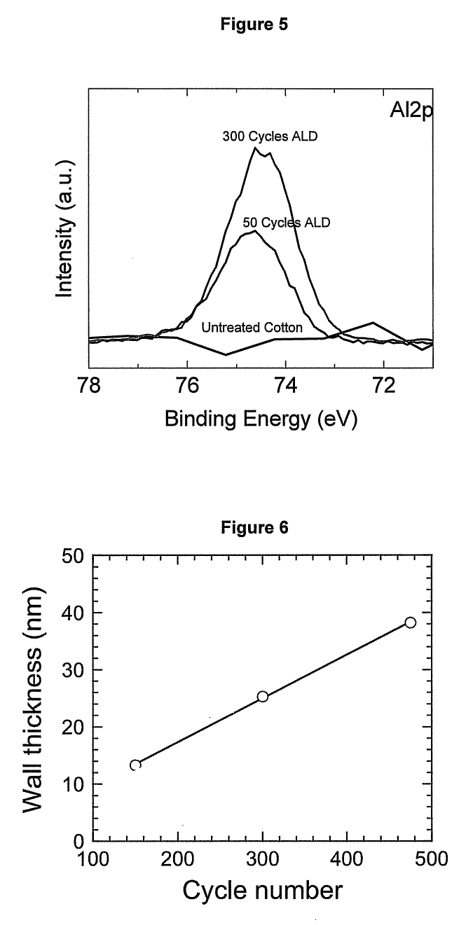

The results in FIG. 4 show the thicknesses of the Al₂O₃ films on the cotton fabric substrates as a function of the number of cycles. The films, as measured by transmission electron microscopy (TEM), were shown to have an initially high growth rate of approximately 5 Å per cycle followed by a decrease to approximately 3 Å per cycle as growth proceeded. X-ray photoelectron spectroscopy (XPS) was used to examine the chemical composition and bonding of the Al₂O₃ films on the cotton substrates. The results in FIG. 5 show the results of an XPS detail scan of Al 2p for untreated cotton and cotton with 50 and 300 cycles of ALD Al₂O₃. No Al peak is observed for the untreated cotton, whereas and Al 2p peak appears at around 74.5 eV after 50 ALD cycles. The intensity of this peak can be seen to increase for 300 ALD cycles. The relative magnitude of the peaks is consistent with the relatively large initial growth per cycle demonstrated in FIG. 4. Static water contact angle measurements showed that a cotton fabric treated with 100 cycles of Al₂O₃ had a contact angle of 127°, compared to a contact angle of 0° for the untreated cotton.

### Example 2

Electrospun polymer fibers of polyvinyl alcohol (PVA) were used as templates for the fabrication of Al₂O₃ microtubes with precisely controlled wall thicknesses using ALD. Coated fibers were created using a hot-wall viscous-flow tube reactor with a reaction temperature of 45° C. and an operating pressure of approximately 0.5 Torr. The aluminum precursor and oxygen reactant used were Al(CH₃)₃ and water, respectively. The TMA and water were introduced into the reaction chamber in an alternating fashion in pulses of 5 seconds and 0.5 seconds, respectively. Argon was pulsed after the introduction of the precursor and reactant in pulses of 20 seconds for the TMA and 60 seconds for the water. The Al₂O₃ coated fibers were heated in air at 400° C. for 24 hours to remove the organic fiber component of the composite structure.

FIG. 6 shows the wall thickness for the Al₂O₃ microtubes as a function of the number of ALD cycles as measured by TEM. The average growth rate of the Al₂O₃ on the fibers is about 0.08 nanometers per cycle. It was demonstrated that ALD of Al₂O₃ can be used to uniformly and conformally coat matrices electrospun PVA fibers. Scanning electron microscopy (SEM) and TEM images provided direct measurements of the uniformity and thicknesses of the deposited films. By varying the electrospinning parameters, the characteristics of the microtubes such as diameter, alignment, and structure, can be tuned. The ALD process can be used to create microtubes of other materials such as TiO₂ and TiN.

### Example 3

Titanium nitride (TiN) thin films were deposited on woven cotton fabric samples using a hot-wall viscous-flow tube reactor at a temperature of 100° C. and an operating pressure of 2 Torr. Tetrakis(dimethylamido)titanium (TDMAT) was used as received from the supplier, and was maintained at 27° C. while being introduced into the reactor using argon as a carrier gas. Argon for both purging and precursor dosing was flowed at 100 sccm. Ammonia was used as the reactant and was introduced into the reaction system at a rate of 100 sccm. The cotton fabrics were placed in the reaction system and heated in vacuum (5×10⁻⁶ Torr) to 100° C. The ALD process used consisted of a five second argon purge, a five second TDMAT dose, a five second argon purge, and a five second ammonia dose.

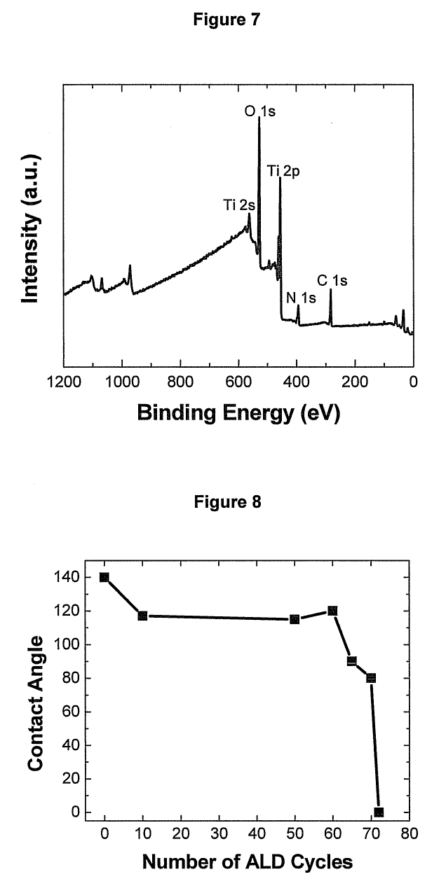

Thicknesses of the TiN films were measured using TEM. The growth rate of the films was approximately 2 A per cycle. XPS measurements, as shown in FIG. 7, demonstrate the deposition of the TiN onto the cotton fabric. The effect of the TiN coatings on the surface energy of the cotton fibers was examined using sessile drop experiments. Static water contact angle measurements show that fabric samples treated with a low number of ALD cycles exhibit very large contact angles, of approximately 122° for fabric treated with 5 cycles of TiN ALD, when compared to untreated cotton. Static contact angle experiments were also used to demonstrate the ability of the ALD process to penetrate through the complex, three dimensional cotton fabrics. A fabric sample was folded during ALD, with the left-half of the sample folded under the right-half of the sample. The right side showed a darker, brownish gold color, except where thermal tape partially covered the surface to hold the sample in place. Both sides of the sample clearly demonstrated hydrophobic behavior, ascribed to TiN deposition throughout the sample volume.

### Example 4

Titanium dioxide (TiO₂) thin films were deposited on woven cotton fabric samples using a hot-wall viscous-flow tube reactor at a temperature of 100° C. and an operating pressure of 2 Torr. Tetrakis(dimethylamido)titanium (TDMAT) was used as received from the supplier, and was maintained at 27° C. while being introduced into the reactor using argon as a carrier gas. Argon for both purging and precursor dosing was flowed at 100 sccm. Water was used as the reactant and was introduced into the reaction system as a vapor at a rate of 100 sccm. The cotton fabrics were placed in the reaction system and heated in vacuum (5×10⁻⁶ Torr) to 100° C. The ALD process included a ten second argon purge, a five second TDMAT dose, a ten second argon purge, and a five second water dose.

XPS measurements demonstrate the deposition of the TiO₂ onto the cotton fabric. Distinct peaks can be seen as a result of the deposition of the thin film of the TiO₂ on the fibers. The effect of the TiO₂ coatings on the surface energy of the cotton fibers was examined using sessile drop experiments. As expected, the untreated cotton fabric samples have a contact angle of 0°. After 1 ALD cycle, the fabric substrates exhibit a contact angle of approximately 113°. This contact angle does not change until more than 25 cycles of TiO2 ALD have occurred. For samples treated with more than 20 cycles of TiO2, the cellulose fibers become very hydrophilic with contact angles of 0°.

### Example 5

Aluminum oxide (Al₂O₃) films were deposited on nonwoven polypropylene substrates in a hot-wall viscous-flow tube reactor at temperatures of 45° C. and 100° C. Trimethylaluminum (TMA) and deionized water were used as precursor and reactant, respectively, for the deposition of Al₂O₃. Reactant lines were heated to 60° C. in order to prevent condensation of the water reactant. Immediately before the deposition process, the substrates were placed in the reactor and heated in vacuum (5×10−7 Torr) to ˜100° C. and allowed to equilibrate for 30 minutes. To begin deposition, the reactor was flushed with argon and ambient temperature vapors of TMA and water were separately introduced into the reactor in pulses of 1 and 0.5 seconds respectively, with a 20 second Ar purge after each precursor exposure step and a 60 second Ar purge after each reactant exposure step. The TMA and water were carried into the reactor using Ar flow, and the Ar flow rate was constant at 100 standard cubic centimeters per minute (sccm). The operating pressure in the reaction chamber was fixed at 0.9 Torr.

The results of static contact angle measurements on polypropylene fabric coated with different thicknesses of Al₂O₃ can be seen in FIG. 8. Static water contact angle measurements showed that the untreated nonwoven polypropylene substrates had a contact angle of approximately 140°. Ten cycles of ALD of Al₂O₃ deposited at 45° C. on the polypropylene substrates resulted in a contact angle of 130°. The static contact angle measurement drops as the number of ALD cycles increases until a critical point is reached at 72 cycles. The contact angle for a sample treated with 70 cycles of ALD of Al₂O₃ is approximately 80° whereas the contact angle for a sample treated with 72 cycles is 0°.

### Example 6

Amine-group functionalized fiber surfaces can be produced directly from the ALD process itself, for example, using ammonia exposures in place of water as the reactant as described in Example 5.

### Example 7

Further amine-group functionalization of the polypropylene substrate produced in Example 6 can be carried out utilizing polyethylene glycol (PEG) groups with amine termination. Specifically, hydroxyl groups present on the substrate from Example 6 will be treated using y-amino-propyltriethoxysilane (APTES) as an anchoring layer followed by attachment of a mini-PEG (Fmoc-NH-(C2H5O)3-COOH, for example, from Peptides International, Inc.). The standard procedure involves sequentially exposing the fibers to the polymers dissolved in non-aqueous solvents. The APTES layer can be prepared by immersing the coated fibers into 1 wt % APTES in anhydrous toluene at 60° C. for 5 minutes followed by a toluene rinse. The functionalized mini-PEG (Fmoc-NH-(C2H5O)3-COOH) can then be linked to the surface using ethyl-3-(3-dimethylaminopropyl) carbodiimide hydrochloride (EDC) as coupling agent with the presence of N-hydroxysulfosuccinimide (Sulfo-NHS) to stabilize the intermediates. The final step involves deprotecting the amino group at the end of the mini-PEG using 50% (v/v) piperidine solution in DMF. Alternate solvents or solvent-free processes can also be considered. The density of amine functionalization can be evaluated using techniques including quantitative bovine serum albumen adsorption and ion-exchange chromatography.

### Example 8

Tests were performed to evaluate ALD processing of Al₂O₃ on PDMS microfluidic channels. The process concept is shown in the schematic in FIGS. 9A-9C. A microfluidic channel formed by conventional PDMS processing is introduced into an ALD reactor system and the process conditions are adjusted to promote reactant and product diffusion into and out of the narrow channel structure. The results show that ALD Al₂O₃ readily coats the PDMS surface, and deposition is clearly observed through entire channels that are sub-millimeter in cross-section and centimeters in length. In principle, the mold could be stripped away leaving a free-standing microfluidic channel as shown in FIGS. 9A-9C, enabling direct integration with other flow structures to create intricate 3D microfluidic networks. The coatings obtained according to this procedure are predicted to perform as high-quality gas barriers that are also impermeable to water and organic solvents.

### Example 9

ALD was used as a process to produce inorganic metallic bio-adhesive coatings on cellulosic fiber substrates. Titanium nitride coatings were deposited on silicon and woven cotton fibres using ALD at 100° C. One cycle of the process included introduction of TDMAT, followed by argon purge, then NH₃ exposure followed by another argon purge. Each gas was pulsed into the reactor for 5 second pulses in the order TDMAT/Ar/NH₃/Ar. The composition of cotton was approximately 97% the polymer cellulose (C₆H₁₀O₅)ₙ, which presents —OH groups on the surface capable of reacting with the initial dose of TDMAT.

Tetrakis(dimethylamido)titanium (TDMAT) was used as received from the supplier, and was maintained at 27° C. while being introduced into the reactor using argon as a carrier gas. Argon dried through a gas drier was used for both purging and precursor dosing, and flow was maintained at 100 standard cubic centimeters per second (sccm) throughout the deposition process. Ammonia was introduced as received into the reactor at the same flow rate as the argon. Pressure was maintained at 2 Torr during processing. Just before the deposition process, substrates were introduced into the reactor and heated in vacuum (5×10⁻⁶ Torr) to 100° C. Each run typically contained cotton samples for contact angle, XPS and TEM measurements, and silicon for profilometry measurement.

X-ray photoelectron spectroscopy (XPS) was used to determine the film thickness and composition on the cotton fabric substrates and transmission electron microscopy (TEM) was used for characterizing film conformality, uniformity, and thickness of the layers on the coated fibres.

Excess adipose tissue from elective plastic surgery procedures was obtained with donor consent. Approximately 50 grams of adipose tissue from a 50 year old Caucasian female was rinsed in phosphate buffered saline (PBS), minced with a scalpel, combined with 50 ml of 0.075% collagenase I, 100 I.U. penicillin/100 μg/mL streptomycin in alpha-modified minimal essential medium (A-MEM with L-glutamine), and incubated at 37° C. on a rotator for 30 minutes. 50 ml of complete growth medium (alpha-modified minimal essential medium (α-MEM with L-glutamine), 10% fetal bovine serum), 100 I.U. penicillin/100 μg streptomycin per mL, 200 mM L-glutamine was added, and the suspension centrifuged for 10 minutes at 10,000×g. The supernatant was discarded, and the hADSC-rich cell pellet suspended in 160 mM NH₄Cl for 10 minutes to lyse red blood cells. Unlysed cells were pelleted by centrifugation for 10 minutes at 10,000×g, and seeded in tissue culture flasks (one 75 cm² flask per 5 grams initial tissue) in complete growth medium. After 24 hours, cultures were washed with PBS to remove non-adherent cells and supplied with fresh growth medium. Cultures were passaged or cryopreserved at 80% confluency. Re-seeding density was 100,000 cells per 75 cm² flask. Cells for this study were used at the third to fifth passage following isolation.

Third to fifth passage cells were grown to 80% confluency, trypsinized, and resuspended in growth medium. Circular pieces of the cotton fabrics with varying TiN coating thicknesses were cut and prewet with phosphate buffered saline (PBS) and placed in 24 multi-well tissue culture plates. 30,000 cells in a volume of 1 mL were seeded onto each sample and incubated at 37° C. with 5% CO². At 6, 12, and 24 hrs post seeding, hADSC-seeded fabrics were removed for viability and proliferation analysis for each treatment. Viability was determined using a Live/Dead Assay Viability Cytotoxicity kit (calcein AM, ethidium homodimer-1) for Mammalian Cells. Live and dead cells were imaged on the fabrics using fluorescent microscopy. Proliferation was determined by quantifying DNA using the DNA binding dye Hoechst 33258 in a microplate based format. Fabrics for each treatment with attached cells were digested overnight at 60° C. in 2.5 units/ml papain from papaya latex in phosphate buffered saline (PBS) with 5 mM EDTA and 5 mM cysteine HCl, then assayed with Hoescht 33258.

Overall, ALD was able to conformally coat the complex surface of the cotton fiber, the coating being very uniform along the fiber surface. All TiN coatings were cytocompatible and allowed some degree of cell adhesion, regardless of coating thickness, at the end of the 24 hour study.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for modifying a surface of a fiber-based substrate comprising:
introducing the fiber-based substrate into a reaction chamber;
pulsing a vapor-phase precursor comprising an organic or an inorganic component into the reaction chamber to create a partial atomic layer of the organic or inorganic component on the fiber-based substrate and create a first by-product species;
purging the reaction chamber to remove excess of the vapor-phase precursor and the first by-product species;
pulsing a vapor-phase reactant into the reaction chamber to complete the formation of an atomic layer of the desired material and create a second by-product species;
purging the reaction chamber to remove excess of the vapor-phase reactant and the second by-product species, and
repeating the pulsing and purging steps until the desired surface modification is achieved.

2. The method of claim 1, wherein the fiber-based substrate comprises natural fibers, synthetic fibers, or both natural and synthetic fibers.

3. The method of claim 2, wherein the fiber-based substrate is selected from the group consisting of cotton fiber, cotton fabric, woven cotton fabric, non-woven cotton fabric, protein-based fiber, polyvinyl alcohol fiber, polyvinyl alcohol fabric, woven polyvinyl alcohol fabric, non-woven polyvinyl alcohol fabric, polyolefin polymer fiber, polyolefin fabric, woven polyolefin fabric, non-woven polyolefin fabric, polyethylene terephthalate fiber, polyethylene terephthalate fabric, woven polyethylene terephthalate fabric, non-woven polyethylene terephthalate fabric, polyamide fiber, polyamide fabric, woven polyamide fabric, non-woven polyamide fabric, acrylic fiber, acrylic fabric, woven acrylic fabric, non-woven acrylic fabric, polycarbonate fiber, polycarbonate fabric, woven polycarbonate fabric, non-woven polycarbonate fabric, fluorocarbon fiber, fluorocarbon fabric, woven fluorocarbon fabric, non-woven fluorocarbon fabric, glass fiber, glass fabric, woven glass fiber, and non-woven glass fabric.

4. The method of claim 2, wherein the fiber-based substrate is non-woven polypropylene fabric, the precursor is trimethylaluminum (TMA), the inorganic component is Al³⁺ and the vapor-phase reactant is H₂O.

5. The method of claim 1, wherein the precursor is tetrakis(dimethylamido)titanium (TDMAT), the inorganic component is Ti²⁺ and the vapor-phase reactant is ammonia.

6. The method of claim 1, wherein the precursor is tetrakis(dimethylamido)titanium (TDMAT), the inorganic component is Ti²⁺ and the vapor-phase reactant is H₂O.

7. The method of claim 1, wherein the fiber-based substrate is a planar surface.

8. The method of claim 1, wherein the fiber-based substrate is a three-dimensional surface.

9. The method of claim 7 or 8, wherein the surface comprises a polymer based surface.

10. The method of claim 9, wherein the polymer-based surface is selected from the group consisting of polyimide, polyethersulfone, cellophane, polydimethylsiloxane, and polytetrafluoroehtylene.

11. The method of claim 1, wherein pulsing a vapor-phase precursor and pulsing a vapor-phase reactant comprise allowing the vapor-phase components to penetrate a bulk of the fiber-based substrate.

12. The method of claim 1, wherein the vapor-phase precursor can be the same or different for subsequent steps of pulsing the vapor-phase precursor; and wherein the vapor-phase reactant can be the same or different for subsequent steps of pulsing the vapor-phase reactant.

13. The method of claim 1, wherein the desired surface modification produces a desired surface energy.

14. A fiber-based substrate having a modified surface created by the method of claim 1.

15. A fiber-based substrate having a modified surface comprising:
a fiber-based substrate; and
a thin film formed on the fiber-based substrate, the thin film being formed by the atomic layer deposition of a precursor comprising an organic or inorganic component and a vapor-phase reactant reactive with the organic or inorganic component;
wherein the thin film modifies the fiber-based substrate to have a desired surface.

16. The fiber-based substrate of claim 15, wherein the fiber-based substrate comprises natural fibers, synthetic fibers or both natural and synthetic fibers.

17. The method of claim 16, wherein the fiber-based substrate is selected from the group consisting of cotton fiber, cotton fabric, woven cotton fabric, non-woven cotton fabric, protein-based fiber, polyvinyl alcohol fiber, polyvinyl alcohol fabric, woven polyvinyl alcohol fabric, non-woven polyvinyl alcohol fabric, polyolefin polymer fiber, polyolefin fabric, woven polyolefin fabric and non-woven polyolefin fabric, polyethylene terephthalate fiber, polyethylene terephthalate fabric, woven polyethylene terephthalate fabric, non-woven polyethylene terephthalate fabric, polyamide fiber, polyamide fabric, woven polyamide fabric, non-woven polyamide fabric, acrylic fiber, acrylic fabric, woven acrylic fabric, non-woven acrylic fabric, polycarbonate fiber, polycarbonate fabric, woven polycarbonate fabric, non-woven polycarbonate fabric, fluorocarbon fiber, fluorocarbon fabric, woven fluorocarbon fabric, non-woven fluorocarbon fabric, glass fiber, glass fabric, woven glass fiber, and non-woven glass fabric.

18. The fiber-based substrate of claim 15, wherein the thin film comprises a biocompatible material.

19. The fiber-based substrate of claim 15, wherein the thin film modifies the fiber-based substrate to have a desired surface energy.

20. The fiber-based substrate of claim 15, wherein the thin film modifies the fiber-based substrate to be operable as a structure selected from the group consisting of a photocatalyst, a sensor material, a catalytic mantle, an active electronic and energy conversion device, a fuel cell, a target-selective nano and biomolecule filtration and separation structure, a tissue engineering scaffold, and an organic-based photovoltaic structure.

21. A method for producing a high density amine-group functionalized surface on a fiber-based substrate comprising:
introducing the fiber-based substrate into a reaction chamber;
pulsing a vapor-phase precursor comprising an inorganic component into the reaction chamber to create a partial atomic layer of the inorganic component on the fiber-based substrate and create a first by-product species;
purging the reaction chamber to remove excess of the vapor-phase precursor and the first by-product species;
pulsing a vapor-phase ammonia or other amine-containing species into the reaction chamber to complete the formation of an atomic layer of the desired material and create a second by-product species;
purging the reaction chamber to remove excess of the vapor-phase ammonia and the second by-product species; and
repeating the pulsing and purging steps until the amine-group functionalized surface of the desired density is achieved.

22. The method of claim 21, further comprising:
treating the fiber-based substrate with y-amino-propyltriethoxysilane (APTES);
attaching a mini-PEG (Fmoc-NH-(C₂H₅O)₃-COOH to the fiber-based substrate; and
deprotecting the amino group at the end of the mini-PEG, wherein the amine-group functionalized surface of the desired density is achieved.

23. A method for producing a uniformly hydrophilic surface on a fiber-based substrate comprising:
introducing the fiber-based substrate into a reaction chamber;
pulsing a vapor-phase precursor comprising an inorganic component into the reaction chamber to create a partial atomic layer of the inorganic component on the fiber-based substrate and create a first by-product species;
purging the reaction chamber to remove excess of the vapor-phase precursor and the first by-product species;
pulsing a vapor-phase reactant into the reaction chamber to complete the formation of an atomic layer of the desired material and create a second by-product species;
purging the reaction chamber to remove excess of the vapor-phase reactant and the second by-product species; and
repeating the pulsing and purging steps until the uniformly hydrophilic surface is achieved.

24. The method of claim 21, 22, or 23, wherein the fiber-based substrate is selected from the group consisting of cotton fiber, cotton fabric, woven cotton fabric, non-woven cotton fabric, protein-based fiber, polyvinyl alcohol fiber, polyvinyl alcohol fabric, woven polyvinyl alcohol fabric, non-woven polyvinyl alcohol fabric, polyolefin polymer fiber, polyolefin fabric, woven polyolefin fabric and non-woven polyolefin fabric, polyethylene terephthalate fiber, polyethylene terephthalate fabric, woven polyethylene terephthalate fabric, non-woven polyethylene terephthalate fabric, polyamide fiber, polyamide fabric, woven polyamide fabric, non-woven polyamide fabric, acrylic fiber, acrylic fabric, woven acrylic fabric, non-woven acrylic fabric, polycarbonate fiber, polycarbonate fabric, woven polycarbonate fabric, non-woven polycarbonate fabric, fluorocarbon fiber, fluorocarbon fabric, woven fluorocarbon fabric, non-woven fluorocarbon fabric, glass fiber, glass fabric, woven glass fiber, and non-woven glass fabric

25. The method of claim 21, 22, or 23, wherein the fiber-based substrate is non-woven polypropylene fabric, the precursor is trimethylaluminum (TMA), the inorganic component is Al³⁺ and the vapor-phase reactant is H₂O.

26. A fiber-based substrate having a high density amine-group functionalized surface produced according to the method of claim 21 or 22.

27. A fiber-based substrate having a uniformly hydrophilic surface produced according to the method of claim 23.

28. A fabric having a high density amine-group functionalized surface.

29. The fabric of claim 28, wherein the fabric is a non-woven fabric.

30. A filter comprising the fabric of claim 29, further comprising a bound affinity ligand.

31. A fabric having a uniformly hydrophilic surface.

32. The fabric of claim 31, wherein the fabric is a non-woven fabric.

33. A method for depositing polymer films on a fiber-based substrate comprising:
introducing the fiber-based substrate into a reaction chamber;
pulsing a vapor-phase reactant comprising an organic monomer into the reaction chamber to create a partial atomic layer of the organic monomer on the fiber-based substrate and create a first by-product species;
purging the reaction chamber to remove excess of the vapor-phase reactant and the first by-product species;
pulsing a vapor-phase co-reactant comprising a complementary organic monomer into the reaction chamber to complete the formation of an atomic layer of the desired material and create a second by-product species;
purging the reaction chamber to remove excess of the vapor-phase co-reactant and the second by-product species; and
repeating the pulsing and purging steps until a desired polymer film is deposited.

34. The method of claim 33, wherein the reactant and the co-reactant comprise an end-group selected from the group consisting of aldehyde, anhydride, amine, ethyne and sulfide.

35. The method of claim 34, wherein the reactant comprising the organic monomer is pyromellitic dianhydride and the co-reactant comprising the organic monomer is phenylene diamine.

36. The method of claim 34, wherein the reactant comprising the organic monomer is phenylene diamine and the co-reactant comprising the organic monomer is phenylene dialdehyde.

37. The method of claim 33, wherein the vapor-phase reactant can be the same or different for subsequent steps of pulsing the vapor-phase reactant; and
wherein the vapor-phase co-reactant can be the same or different for subsequent steps of pulsing the vapor-phase co-reactant.

38. A fiber-based substrate created by the method of claim 33.

39. A fabric having a high density amine-group functionalized surface, the fabric comprising:
a fiber-based substrate; and
an amine-group functionalized surface formed on the fiber-based substrate, the surface being formed by the atomic layer deposition of a vapor-phase precursor comprising an inorganic component and a vapor-phase ammonia.

40. The fabric of claim 39, wherein:
the fiber-based substrate is treated with y-amino-propyltriethoxysilane (APTES);
a mini-PEG (Fmoc-NH-(C₂H₅O)₃-COOH is attached to the fiber-based substrate; and
the amino group at the end of the mini-PEG is deprotected.

41. The fabric of claim 39, wherein the fabric is a non-woven fabric.

42. The fabric of claim 41, wherein the non-woven fabric comprises a bound affinity ligand.

43. A modified fiber-based substrate comprising:
a fiber-based substrate; and
a polymer film formed on the fiber-based substrate, the polymer film being formed by the atomic layer deposition of a vapor-phase reactant comprising an organic monomer and a vapor-phase co-reactant comprising a complementary organic monomer.

44. A method for depositing a hybrid organic-inorganic film on a fiber-based substrate comprising:
introducing a fiber-based substrate into a reaction chamber;
pulsing a vapor-phase reactant comprising a first component comprising an organic component or an inorganic component into the reaction chamber to create a partial atomic layer on the fiber-based substrate and create a first by-product species;
purging the reaction chamber to remove excess of the vapor-phase reactant and the first by-product species;
pulsing a vapor-phase co-reactant comprising a second component comprising an organic or an inorganic component depending on the first component into the reaction chamber chamber to complete the formation of an atomic layer of the desired material and create a second by-product species;
purging the reaction chamber to remove excess of the vapor-phase co-reactant and the second by-product species; and
repeating the pulsing and purging steps until the desired thickness of hybrid films is deposited.

45. A method for forming a free-standing micro- or nanostructure comprising:
introducing a fiber core into a reaction chamber;
pulsing a vapor-phase precursor comprising an inorganic monomer into the reaction chamber to create a partial atomic layer of the inorganic monomer on the fiber-based substrate and a first by-product species;
purging the reaction chamber to remove excess of the vapor-phase precursor and the first by-product species;
pulsing a vapor-phase reactant into the reaction chamber to complete the formation of an atomic layer of the desired material and create a second by-product species;
purging the reaction chamber to remove excess of the vapor-phase reactant and the second by-product species;
repeating the pulsing and purging steps until a desired thickness of a micro- or nanostructure is deposited; and
removing the fiber core.

46. A free-standing micro- or nanostructure formed according to the method of claim 45.

47. A micro- or nanostructure of claim 46, wherein the micro- or nanostructure is porous.

48. A method for preparing a micro- or nanostructure, the method comprising:
providing a mold comprising a micro- or nanostructure;
introducing the mold into an atomic layer deposition (ALD) reactor system;
adjusting ALD process conditions to promote ALD reactant and product diffusion into and out of the mold, wherein a micro- or nanostructure is formed; and
removing the mold.

49. A method of claim 48, wherein providing a mold comprises providing a mold comprising a micro- or nanostructure by polydimethylsiloxane (PDMS) processing.

50. A method of claim 48, wherein the mold comprising a micro- or nanostructure comprises a microfluidic channel.

51. A method of claim 48, wherein the micro- or nanostructure formed comprises an Al₂O₃ based microfluidic structure.

52. A micro- or nanostructure produced by the method of claim 48.
